(19) United States
(12) Patent Application Publication
(10) Pub. No.: US 2022/0071793 A1
(43) Pub. Date: Mar. 10, 2022

(54) Garment

(71) Applicant: Alignmed, Inc., Santa Ana, CA (US)

(72) Inventor: William J. Schultz, Newport Beach, CA (US)

(21) Appl. No.: 17/384,695

(22) Filed: Jul. 23, 2021

Related U.S. Application Data
(63) Continuation of application No. 17/228,595, filed on Apr. 12, 2021.
Application No. 17/228,595 is a continuation of application No. 17/177,109, filed on Feb. 16, 2021.
Application No. 17/177,109 is a continuation of application No. PCT/US20/14844, filed on Jan. 23, 2020.
Application No. PCT/US20/14844 is a continuation of application No. PCT/US20/13970, filed on Jan. 17, 2020.
Application No. PCT/US20/13970 is a continuation of application No. PCT/US19/55740, filed on Oct. 10, 2019.
Application No. 17/177,109 is a continuation of application No. PCT/US19/55740, filed on Oct. 10, 2019.
Application No. PCT/US19/55740 is a continuation of application No. PCT/US19/46430, filed on Aug. 13, 2019.
(60) Provisional application No. 62/796,035, filed on Jan. 23, 2019.
(60) Provisional application No. 62/744,002, filed on Oct. 10, 2018.
(60) Provisional application No. 62/718,350, filed on Aug. 13, 2018.
(60) Provisional application No. 62/718,353, filed on Aug. 13, 2018.

(51) Int. Cl. A61F 5/02

(52) U.S. Cl. CPC A61F 5/026

(57) ABSTRACT

Exemplary embodiments contained herein include garments having biomechanical components including panels for influencing the wearer. Exemplary embodiments contained herein include garments having features for improving comfort of the wearer during use.

### BACKGROUND

The human form has evolved into an extraordinary structure of bones, muscles and joints to support the upright, moving and thinking body. It is well established in medical science that muscle balance and postural alignment of this body will largely determine how we look, feel, perform, fight pain and manage injury throughout life. All people would benefit from a simple means to train, retrain and maintain muscle and joint balance against the adverse effects of our environment, occupation, injury, disease and aging. A dynamic anatomic garment would provide an interface of therapeutic support and resistance training to a body in static and dynamic motion, and thus passively intervene and influence improvements in wellness and avert the physical maladies associated with injury, arthritis, chronic pain, sensory processing disorders, joint misalignment and poor posture.

Current upper body performance apparel uses compression of skin and muscle, simultaneously or in gradients, as the active mechanism. However, the physiologic function of muscles tissue is to contract in a kinetic sequence as a response to external stimuli, i.e., muscles don't compress, they contract. Compressing skin and muscle does not offer an optimal means to influence improvements in the balance of overused, underused, or otherwise injured muscles and joints that lead to postural abnormalities. Alternatively, performance apparel may include external taping or straps oriented and attached to a garment to impose an external force on the wearer. Integral to the function of such garments is a form-fit that comfortably envelopes a body in motion.

However, inherent in many of the configurations of present garments is pressure and friction that creates discomfort, especially around sensitive areas of the body, including the stomach, breasts, armpits, neck, and other areas of the body. The form fitting nature of a garment, the movement of the body, and any imposed force on the garment generally pulls the garment against the sensitive areas.

### SUMMARY

Exemplary embodiments provided herein includes the construction of bio-ergonomic apparel using elastomeric fabrics with properties of stretch in multi-directional plains that address the human body as a bio-ergonomic workstation with bio-ergonomic apparel that includes garments configured to accommodate the body ergonomics to comfortably conform to the wearer's body in static and dynamic motion. Exemplary embodiments may be used to comfortably conform a garment to different individual wearer's having a wide variation in body sizes, configurations and shapes.

Exemplary embodiments described herein may be used to produce kinetic support and tensile resistance onto targeted muscle tissue for the purpose of training and maintaining muscle balance, joint alignment and good posture. Exemplary embodiments described herein include apparel having variable elasticity and elastic memory properties, placed onto specific anatomy, for the purpose of tensile resistance, which is different from conventional compression or gradient compression garments. Exemplary embodiments provided herein provide panels and seams having different and/or specific elastic force between two or more adjoining panels. The panels and corresponding seams are positioned on the garment to correspond to desired muscle and/or anatomical locations and underlying anatomical physiologic functions. features of the body.

Exemplary embodiments described herein include features, components, and configurations of a garment to relieve discomfort of a wearer during use. The exemplary embodiments may be used in any combination as provided herein. Features may be incorporated into other garments, such as compression garments, taping garments, active wear, or other garments to relieve discomfort. Exemplary embodiments of discomfort reducing features may include any combination of a lower front expansion panel, an upper front expansion panel, a support panel, an armpit force dispersion configuration, sleeve configuration to rotate forces out of the armpit, and other features provided herein.

Exemplary embodiments described herein include garments worn by persons for different purposes including protection from environmental elements, occupational requirements, modesty, adornment, fashion, support, influence neuromuscular activity for therapeutic results, or improve physical orientation. An example is a form-fit garment, worn to influence improvement in overused and underused and thus aid muscle balance, body alignment, circulation and/or postural fitness. A form-fit garment may be used as an adjunctive treatment for repetitive stress injury, chronic pain, chronic fatigue, injury, disease and disorder.

Exemplary embodiments relate generally to a garment or device worn by a person for functional improvements in postural form, health and fitness, comfort, range of motion, reduced interference with range of motion, and combinations thereof.

Exemplary embodiments described herein include different design shapes for garments. The garments may combine comfortable, form-fitting posture garments for the upper extremity. The posture garments may have built in attachment points which are anatomically positioned to accept panels of various elastic properties, shapes, tensile resistance and tactile feel. The panels may be attached to the garment or integrated therein.

Exemplary embodiments may include a wearable device, such as a garment, defining a non-linear and/or non-orthogonal construction of anatomic apparel portions used to systematically interact and influence the innate processes of human physiology for improvement in health and fitness.

Exemplary embodiments may comprise dynamic materials with specific elastic resistance. The dynamic materials may be used to apply dynamic tensile resistance and tactile touch to prevent or reduce muscle atrophy by actively enforcing natural movement and neutral anatomy.

Exemplary embodiments may comprise dynamic materials with specific elastic properties and supple constructions that allow the garments to be comfortably worn under or over clothing without compromising function. The use of the garments over or under clothing greatly improves the use of the garments over desirable periods of time, with little discipline or change in behavior required.

Exemplary embodiments may comprise dynamic materials with specific light weight, breathable properties and UV resistance, which enforce comfort over long-term wear, during sleep, or systematic wear correlated to therapeutic needs of the user.

### DRAWINGS

FIG. 1A illustrates an exemplary garment according to embodiments described herein.

FIG. 1B illustrates the garment of FIG. 1A overlayed with the underlying muscle groups of an exemplary wearer in an in use position.

FIG. 2 illustrates an exemplary garment according to embodiments described herein.

FIGS. 3A-3D illustrate an exemplary garment according to embodiments described herein.

FIGS. 4A-9 illustrate exemplary garments having different configurations of arm aperture for reducing forces directed at the armpit of a wearer.

### DETAILED DESCRIPTION

In the following description of preferred embodiments, reference is made to the accompanying drawings which form a part hereof, and in which it is shown by way of illustration specific embodiments in which the invention can be practiced. It is to be understood that other embodiments can be used and structural changes can be made without departing from the scope of the embodiments of this invention.

Exemplary embodiments described herein use different configurations of elastic force between two or more adjoining panels. Exemplary embodiments may include procedures and techniques that use panels of variable stretch characteristics to mirror the innate physiology of the related muscle to which the band is positioned over. Exemplary embodiments described herein are in terms of panels, but the invention is not limited to a specific geometric configuration or material property of a “panel”. Instead, any configuration of a material portion having the desired relative stretch characteristics and/or positions relative to the body are encompassed within the definition of “panel”. Accordingly, panels may include linear or elongated pieces, circular pieces, and any combination of geometric or non-geometric portions as described herein or would be understood by a person of skill in the art. Panels may also include material sections of a garment, seams, overlayed or multiple layered sections, or other portion of the garment having specific elastic properties.

In an exemplary embodiment, the described procedures and techniques may include positioning and orienting panels such that they are anchored to start and end in line with or over corresponding neuromuscular anatomical features. The configuration and position may therefore mirror or correspond to neuromuscular kinesiology or neuromuscular mapping. Exemplary embodiments of panels may be used to physically facilitate muscles and their function to support, load, and unload joints. Exemplary embodiments may be used to position joints in certain positions that are in line with good posture and optimal body mechanics. Exemplary embodiments may be used to enhance posture, reduce pain, and aid in recover.

Exemplary embodiments described herein include a wearable device. The wearable device may use non-restrictive, anatomically fit, elastomeric (stretchable) materials configured and attached together corresponding to anatomic anchoring locations, and having material fiber orientations in-line with contractual properties of the muscles where the material sections overlay or link together, and combinations thereof. When worn, exemplary embodiments of a garment having features described herein may be used to interact with the shape and motion of the wearer to generate and/or deliver therapeutic modalities including, but not limited to, tactile biofeedback, muscle support, tensile resistance used to train, retrain, maintain, or otherwise improve the wearer's muscle balance, circulation, physical appeal and overall postural fitness. Exemplary embodiments may be used for muscle and postural training when worn intermittently.

Exemplary embodiments may be used with garments having a form fit, such as performance apparel, compression apparel, shapewear, maternal wear, among others. Such exoskeletal apparel may produce atypical tensions and pressures which can be restrictive, ill-fitting, ill-positioned, or uncomfortable. Exemplary embodiments may be used to improve conform and anatomic conformability using construction techniques and materials that address the innate properties of human anatomy and physiology. Exemplary embodiments may use, configure, and position materials and component panels to synergistically assist or correspond with contractual properties of the muscle groups in which they overlay, contact, or traverse. Exemplary embodiments may use, configure, and position materials and component panels to direct forces and portions of the garment away from sensitive areas during normal use and contraction and movement of the garment. Exemplary embodiments may permit independent movement, expansion, or orientation of one panel of the garment separate from another panel or portion of the garment. Exemplary embodiments may assist or correspond with contractual properties of the muscle groups in which they overlay, contact, or traverse. Exemplary embodiments may configure material sections to define or correspond to an anatomical form having boarders and/or seams corresponding to anatomical anchoring locations to address the kinetic sequence of the muscle tissue they adjoin.

Exemplary embodiments may incorporate stretch and tensile characteristics that are placed directionally and in line with human kinetics. Exemplary embodiments may configure material sections to define or correspond to an anatomical form having boarders and/or seams corresponding to anatomical anchoring locations to address the kinetic sequence of the muscle tissue they adjoin.

Exemplary embodiments include using fabric fiber orientation and bias of stretch within a material as a mechanism of action to provide neurobiofeedback to the wearer. The use of fiber orientation within exemplary embodiments may provide a precision based application of tensile resistance when placed in the kinetic direction of muscle force.

Exemplary embodiments include a garment and method that actively influences the body's own resources to preserve and maintain optimal alignment, that uses biofeedback to enhance the senses of proprioception, kinesthesia, touch and other components of the central, peripheral and somatosensory nerves to maintain and preserve good posture, that uses biofeedback to support optimal body mechanics while simultaneously enhancing muscle memory and muscle retraining, and combinations thereof.

Exemplary embodiments may include panels having synthetic and/or natural materials with stretch characteristics designed to mimic the contractual properties of muscle tissue for which the panel overlays. Exemplary panel construction and fiber orientation can be adjusted to assist specific muscles and joints to perform specific functions. Exemplary panels may be constructed to allow their forces to be spread over a broad area. Exemplary panels may include tensile and tactile properties configured to dynamically provide biofeedback to facilitate muscle and joints movement in the performance of human mobility. Exemplary panels may be kinetically placed and may be adjusted to assist muscles and joints in performing specific functions.

Exemplary embodiments may include garments constructed to avoid compression in one or more areas of the body. For example, exemplary garments may include expansion panels configured to permit less restrictive movement or reduce pressure on the underlying anatomic features of the body. Exemplary expansion panels may be included over or around the abdomen, neck, thorax, armpit, other soft tissue areas, and combination thereof. Exemplary expansion panels may include bi-directional or multi-directional stretch orientations to provide greater expansion of the panel over select areas of the body

Exemplary embodiments of a garment include exemplary seam constructions to coordinate specific elasticity and tensile support throughout the garment. Exemplary seam constructions and placement may correspond to human kinetics and directional transfer of tensile strength of associated panels to which the seam may be coupled.

Exemplary embodiments may include material portions having unidirectional, bidirectional, multi-directional bias of elasticity. Exemplary embodiments may orient the bias of elasticity in specific configuration and orientations to support the underlying body anatomy according to embodiments described herein. Exemplary embodiments may include form fitting garments having a front portion, a back portion, and arm portions. The exemplary garment may include lateral side portions that are incorporated into the front and/or back portions or may be separate thereto.

As used herein, elasticity generally refers to the amount a material can stretch under a given force. Therefore, a greater elasticity indicates a greater amount of stretch when a force is imposed thereon. Rigid or semi-rigid materials are considered to have low elasticity and do not stretch or only include a very limited stretch under an imposition of force. As used herein, the bias of stretch generally indicates that the material has a greater elasticity in the direction of the bias of stretch than in other directions. A material may have one or more than one bias of stretch. Each direction of a material's bias of stretch may have the same elasticity or different elasticities. Therefore, a material may have a major bias of stretch in a first direction and a second bias of stretch in a second direction, where the first bias of stretch has a greater elasticity than the elasticity associated with the second bias of stretch. In an exemplary embodiment, the bias of stretch in the first direction and second direction may define different axis of bias of stretch. The major axis of bias of stretch associated with a maximum bias of stretch direction may be in a first direction. The minor axis of bias of stretch associated with a greater elasticity in a direction than other directions, but which may be less than or equal to the elasticity of the bias of stretch along the major axis of bias of stretch.

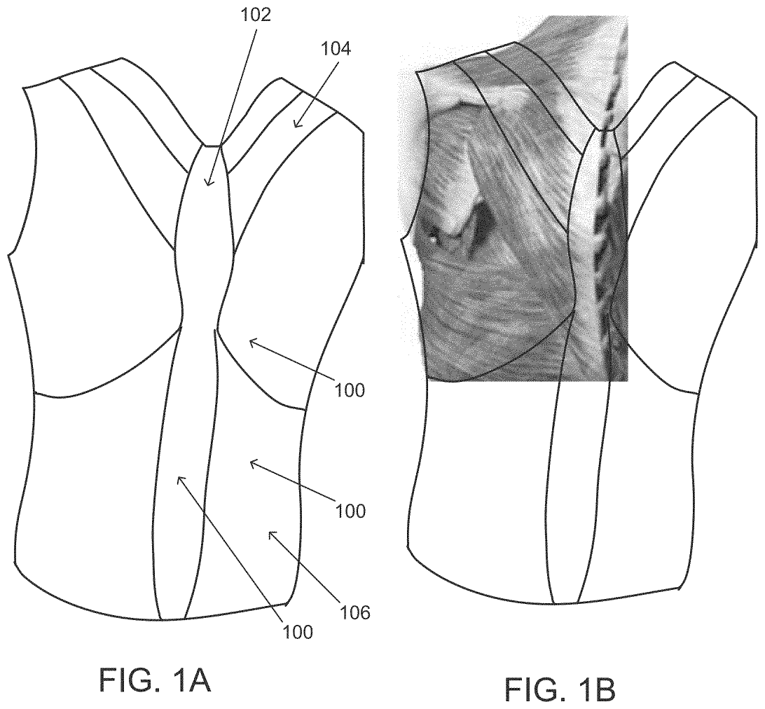

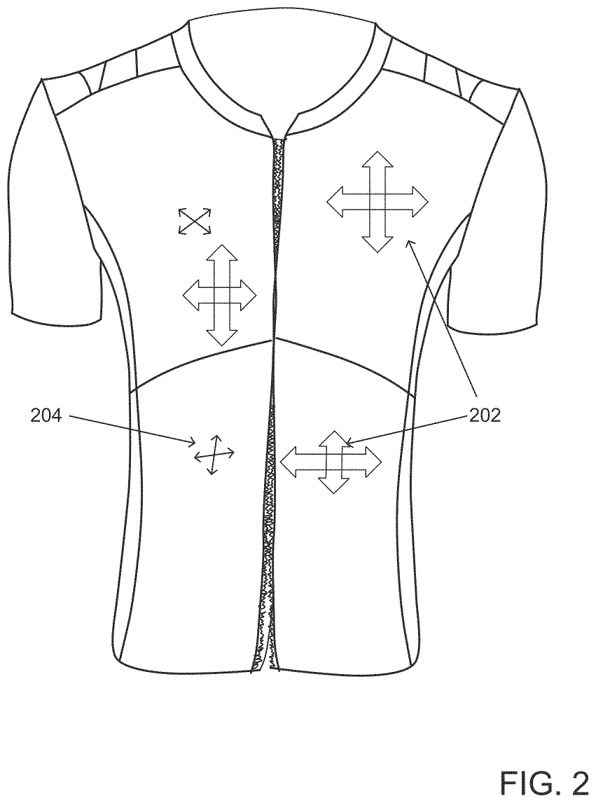

FIG. 1A illustrates an exemplary back side of a garment according to embodiments described herein made up of a plurality of panels 100. FIG. 1B illustrates an exemplary illustration of the garment of FIG. 1A overlaid upon an exemplary muscle groups to illustrate the corresponding muscle tissue for which the panels overlay. The panels may include different elastic properties that facilitate movement with the underlying muscle groups. Exemplary embodiments described herein include apparel having variable elasticity and elastic memory properties, placed onto specific anatomy, which is different from conventional compression or gradient compression garments. Exemplary embodiments provided herein provide panels 100 having different elastic properties between two or more adjoining panels. The panels and corresponding seams are positioned on the garment to correspond to desired muscle and/or anatomical locations and underlying anatomical features of the body.

For example, as seen in FIG. 1A, a first panel 102 may extend along the middle region of the garment from the neck aperture to the bottom aperture of the garment. The first panel 102 may be configured to overlay a spine of the wearer in a worn position. The first panel 102 may be along the length of the garment along the axis of symmetry of the garment on the back side of the garment. A second panel 105 may extend from the first panel 102 over the shoulder region of the garment from the back of the garment to the front of the garment over a top of the garment, adjacent the neck aperture of the garment. The second panel 105 may extend between the neck aperture and the arm aperture or sleeve of the garment. The second panel may include one, two, three, or more similar panels extending from the first panel 102 on the back of the garment to the front of the garment. Each second panel 104 may be separated from an adjacent second panel 104 if more than one second panel is positioned on the same side of the garment from the first panel 102 over the same shoulder of the garment. Each of the second panels 104 may have the same elastic property, while sections positioned between adjacent panels may have different elastic properties. In an exemplary embodiment, the elasticity of the second panels 104 is greater than the elasticity of the first panel 102. Other panels may be included having the same or different elasticities.

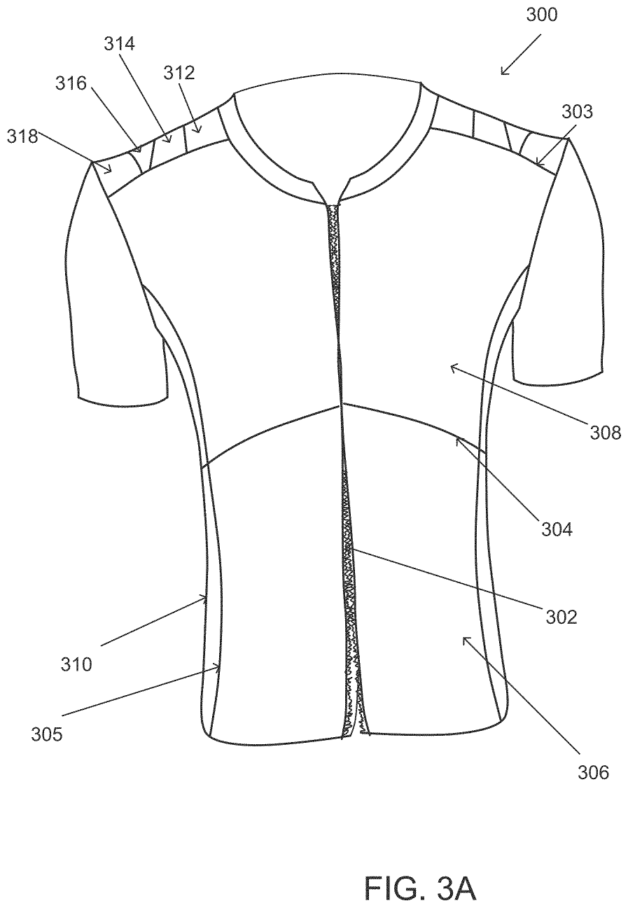

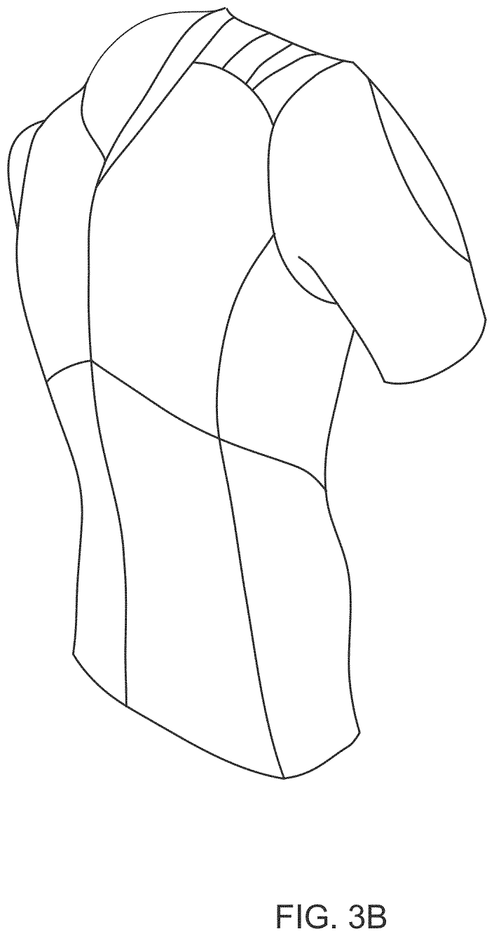

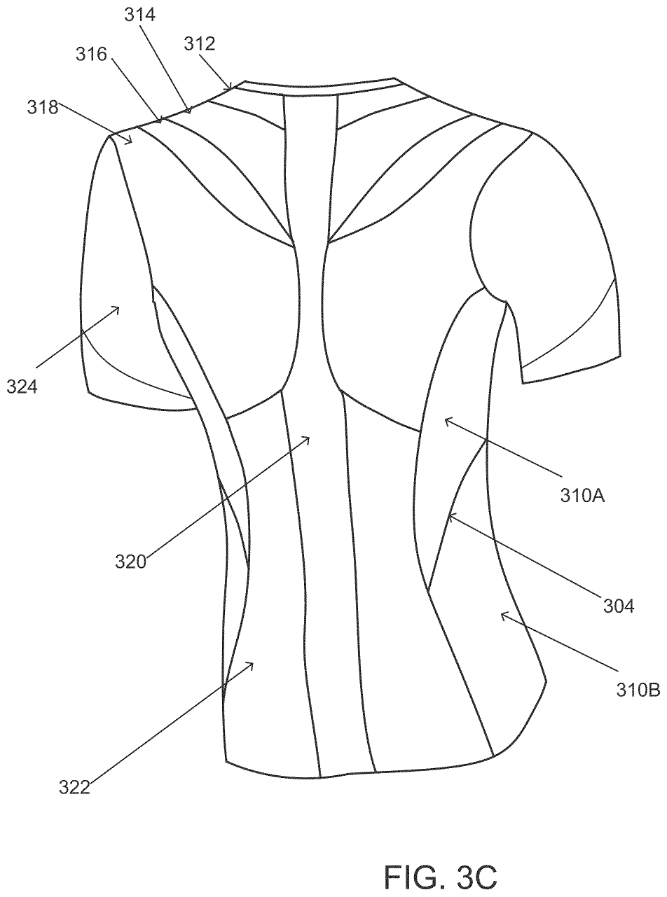

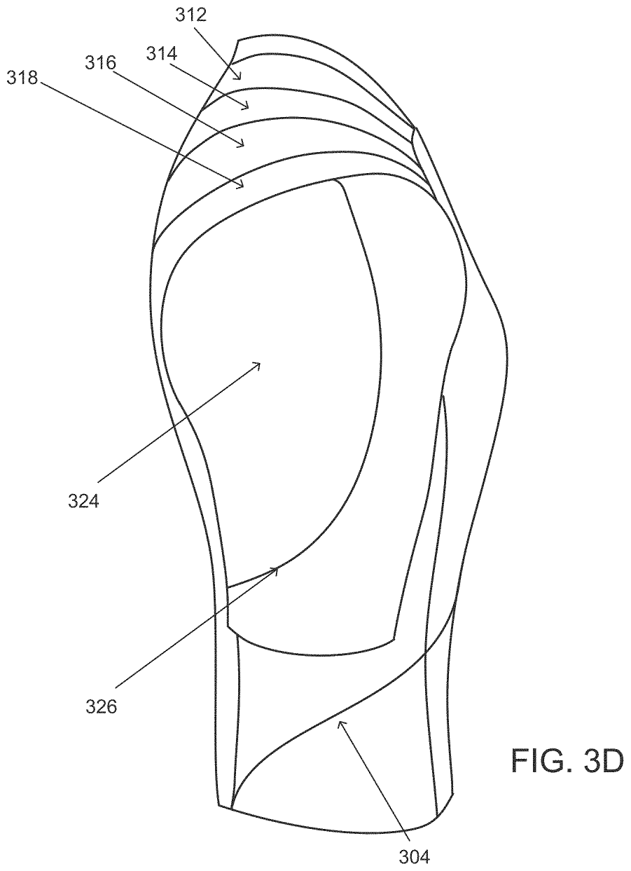

FIGS. 3A-3D illustrate an exemplary garment according to embodiments described herein.

FIG. 3A illustrates a front portion of the garment having a plurality of panels having different elasticities. FIG. 2 illustrates an exemplary front portion of the garment illustrating exemplary elasticity and fiber orientations of the exemplary panels. As illustrated a portion 304 of the garment may divide adjacent panels 306,308. The dividing portion 304 may create an area of the garment having a reduced elasticity relative to one, both, or all of the elasticities of the panels 306, 308 coupled or adjacent thereto. The dividing portion 304 may acts as an anchor or separator of the garment, such that the panels 306,308 on opposing sides of the dividing portion 304 may stretch, move, and conform to the individual wearer without or with reduced impact on the other panels. The panels adjacent to the dividing portion 304 and on opposing sides thereof, may have different elasticities to accommodate different body configurations, comfort of the wearer, movement of the wearer, or other benefits and purposes.

The front of the garment may include a vertical zipper 302 extending from the top of the garment at the neck aperture to the bottom of the garment. The zipper may extend along a center axis defining a longitudinal axis of the garment. In an exemplary embodiment, the zipper is a bi-directional zipper that permits a wearer to open and/or close the zipper from both ends of the zipper. In this way, the garment may additionally facilitate different body configurations and different dynamic conditions of the wearer.

As illustrated in FIG. 3A, a front of a garment may include an upper panel 308 and a lower panel 306. The upper panel 308 may accommodate expansion or different configurations of body styles, such as various soft tissue sizes of the chest. Similarly, the lower panel may also accommodate expansion or different configurations of body styles, such as the variable size and expansion of the stomach area of the wearer.

As illustrated in FIG. 2, the upper panel 308 may have a bias of elasticity illustrated by the outlined arrows 202. The bias of elasticity may be bi-directional, and the elasticity may be the same or different along the axis of bias. As represented in FIG. 2, the bias of elasticity may be the same in two directions as represented on the right side of the garment, or may include a major axis and minor axis of bias as represented on the left side of the garment. The two sides of the garment may include the same configuration of bias of stretch on opposing lateral sides of the garment, as the representations of bias of stretch are exemplary of different configurations only. As illustrated, the upper panel may have a bias of stretch in a first direction and a second direction that are approximately equal. The upper panel may also have a major bias of stretch that is more than a minor bias of stretch by greater than zero to fifty percent more stretch. In an exemplary embodiment, the major bias of stretch is approximately zero or greater than zero to thirty percent more than the minor bias of stretch. As illustrated, the major bias of stretch may be oriented in a vertical direction from the top of the garment to the bottom of the garment. The minor bias of stretch may be across the garment in a horizontal direction from/to lateral sides of the garment. In an exemplary embodiment, the stretch factor of the top upper panel may be designated as a 50/50, 60/40, 70/30. These ratios indicate the amount of stretch in the major axis verses the minor axis.

As illustrated in FIG. 2, the lower panel 306 may have a bias of elasticity illustrated by the outlined arrows 202. The bias of elasticity may be bi-directional, and the elasticity may be the same or different along the axis of bias. The lower panel may have a bias of stretch in a first direction and a second direction that are approximately equal. As illustrated, the lower panel may have a bias of stretch in a first direction greater than a second direction. The lower panel may also have a major bias of stretch that is more than a minor bias of stretch by greater than zero to fifty percent more stretch. In an exemplary embodiment, the major bias of stretch is approximately zero or greater than zero to thirty percent more than the minor bias of stretch. As illustrated, the minor bias of stretch may be oriented in a vertical direction from the top of the garment to the bottom of the garment. The major bias of stretch may be across the garment in a horizontal direction from/to lateral sides of the garment. In an exemplary embodiment, the stretch factor of the lower panel may be designated as a 50/50, 60/40, 70/30. These ratios indicate the amount of stretch in the major axis verses the minor axis.

The bias of elasticity of the lower panel 306 may be different than the upper panel 308. As illustrated, the major axis of bias may be different between the upper panel and the lower panel. The major bias of elasticity of the upper panel to the lower panel may be oblique or perpendicular to each other. In an exemplary embodiment, the major axis of bias of the upper panel is vertical, while the major axis of bias of the lower panel is horizontal.

The upper panel 308 and lower panel 306 may be coupled and divided by a dividing portion 304. The dividing portion 304 may be a seam, integration of adjacent panels, bond, another panel, or combinations thereof. The dividing portion 304 may have a reduced elasticity as compared to the upper panel 308, lower panel 306, or both panels. As illustrated, the dividing portion 304 is positioned transversely across the front of the garment. The dividing portion 304 may be arched/curved to conform to anatomical features underlying the dividing portion. For example, the dividing portion 304 may be positioned to correspond or overlay the rib cage area of a wearer in an in use position. The position may approximate the lower ribs, such as the last rib bone. The dividing portion 304 may start at approximately a center area of the garment, and extend on opposing lateral directions toward the lateral sides of the garment, under the arm apertures or sleeves of the garment. The dividing portion 304 may curve from the maximum height toward the neck aperture toward a lower end of the garment as the dividing portion 304 extends toward the lateral sides of the garment, such that the curve created by the dividing portion 304 is concave downward. The seam may approximate the position of the last rib bone and be positioned over the muscle groups corresponding to the external oblique aponeurosis, laterally across the transverse abdominis, and the outer lateral side of the external oblique abdominal aponeurosis, and toward the latissimus dorsi muscle.

The front of the garment may also include lateral dividing portion 305. The lateral dividing portion 304 may be a seam, integration of adjacent panels, bond, another panel, or combinations thereof. The dividing portion 305 may have a reduced elasticity as compared to the upper panel 308, lower panel 306, or both panels. As illustrated, the lateral dividing portion 305 is positioned vertically and extends along the lateral side of the garment. The lateral dividing portion 305 may be positioned outside of the stomach region when the garment is in an in use position. The lateral dividing portion 304 may be used to anchor and/or position the garment relative to the wearer during use, such that expansion of the lower and/or upper panels does not adversely affect or minimizes the repositioning of the rest of the garment.

The garment may also include lateral side panels 310. The lateral side panel may extend from under the armpit, arm aperture, or sleeve, to the lower end of the garment. A lateral side panel 310 may be positioned on each of opposing sides of the garment. The lateral side panel 310 may be in two portions, including a first lateral side panel 310A corresponding or adjacent to the upper panel 308, and a second lateral side panel 310B corresponding or adjacent to the lower panel 306. The lateral side panel may be a single panel or multiple panels coupled together. The lateral side panel may include an extension of the dividing portion 304 extending continuously there through, or the dividing portion 304 may terminate at the lateral side panel. The dividing portion 304 may therefore extend from the lower rib cage on the front, middle of the garment around the lateral side to approximately the position of the thoracolumbar fascia when the garment is in an in use configuration. The dividing portion 304 may follow the iliac crest over the hip of the wearer in an in use configuration. In an exemplary embodiment, the dividing portion 304 may extend all of the way to the support panel 320 described herein. As illustrated, the lateral side panel coupled the front panels of the garment to the back panels of the garment. The lateral side panel may have a lower elasticity than the lower front panel 306. The lateral side panel may have a lower or equal elasticity than the upper front panel 308. The lateral side panel may be of the same or different material than the lower panel 306, and/or upper panel 308.

The garment may also include a back portion having a plurality of panels according to embodiments described herein. The back portion of the garment may include support panels 314, 318, 320. The support panel 320 is positioned and extends along a spine of the wearer in an in use configuration. The support panel 320 is centrally located and extends from the neck aperture to the bottom of the garment along and defining the longitudinal axis of the garment. The support panel 320 may have a variable width along its length. As illustrated, the support panel 320 is narrower and curved inward at a middle portion of the support panel 320. The narrowing of the support panel 320 occurs between elastic panels 318, and corresponds to the middle of the back, approximate the level of the shoulder blades or lower, such as at the level of the infraspinatus fascia, when the garment is in the in use configuration. The support panel 320 may be less elastic than one, more, or all of the panels of the garment, excluding dividing portions. The support panels 314 extend from the central support panel 320 up and over the shoulder of the wearer and up and over the portion of the garment from the back of the garment to the front of the garment between the space between the neck aperture and the arm aperture/sleeve. The support panel 314 may have a greater width along the center support panel 320 and then taper as the support panel 314 extends over the shoulder of the garment. The support panel 314 may extend to the front of the garment. The support panel 314 may end approximate the collar bone of the wearer when the garment is in an in use position. The support panel 314 may terminate at a dividing portion 303 extending between the neck aperture or neck area and the sleeve or arm aperture. The dividing panel 303 may extend approximately along the collar bone of the wearer in an in use configuration of the garment. The other support panel 318 may extend along the lower edge to approximately the teres minor muscle and along the upper edge or portion of the latissimus dorsi muscle. The top portion of the other support panel 318 may similar extend upward and outward from the central support panel 320 over the shoulder area of the garment between the neck aperture and arm aperture/sleeve of the garment. The support panel 218 may therefore be positioned to overlay the teres major, teres minor muscle groups and extend along the spine of scapula over the shoulder of the wearer.

The garment may include panels to facilitate breathability, moisture wicking, bacterial resistance, other purposes, and combinations there of. In an exemplary embodiment, the garment may include panels 322, 316, 312 positioned between support panels 320 and elastic panels 318. These panels may include mesh to facilitate air flow and breathability of the garment. These panels may have a greater elasticity than the support panels. These panels may be used to stretch the garment to accommodate users of variable configuration, size, and body structure. For example, a user with wider hips will impose a lateral force on the garment, in which the panels 322 can accommodate, while maintaining the support panels 320 in a desired location relative to the body anatomy of the wearer.

In an exemplary embodiment, the garment sleeve is made of a single panel having only a single, continuous seam coupling the sleeve to the garment and to itself. The exemplary garment defines a curved seam that extends along and proximate the shoulder blade of the wearer in a worn position. For example, a portion of the seam may overlay the bone of the shoulder blade, the infraspinatus fascia, and outward along the spine of scapula. The seam may define a support position and may overlay anatomical attachment areas of the muscle groups to the body, such as over fascia or bone. The top of the sleeve seam may be positioned in line with the deltoid fascia when the garment is in an in use configuration.

In an exemplary embodiment, the sleeve has a first end and a second end with apertures there through for passing an arm of a wearer. The first end may be directly attached to the garment, such as along front, back, and lateral sides. The sleeve may taper from the first end to the second end. In an exemplary embodiment, the sleeve is attached at a seam from the first end to the second end. The seam is curved along an entire length from the first end to the second end. In an exemplary embodiment, seam 326 starts at the top of the garment over the shoulder, between the sleeve and the neck aperture. In an exemplary embodiment, the seam 326 starts posterior to the acromioclavicular joint. The seam then extends toward the front of the garment to spiral around toward the pectoral of the wearer before curving backward as the seam extends downward toward the sleeve aperture. The seam may follow the bicep/triceps split and spiral posterior around the back of the sleeve.

In an exemplary embodiment, the sleeve may have its own bias of elasticity. As illustrated, the sleeve includes a bidirectional bias of elasticity. The sleeve wraps around to create the sleeve and define the passage for the arm therethrough. The same bias of elasticity may wrap or spiral around the sleeve, such that the same bias of elasticity may have a component directed along and around the circumference of the sleeve as well as a component directed along the length of the sleeve.

Garments according to embodiments described herein may include the same sleeve configuration and/or length on both sides of the garment. Exemplary embodiments of a sleeved garment may include a sleeve of any length. A short sleeve and long sleeve are illustrated for sake of example, but other sleeve lengths, such as ½ or ¾ sleeves are also within the scope of the present description.

Exemplary embodiments of the sleeve, sleeve aperture, lateral panels, and combinations thereof may be configured to disburse the forces of the garment away from the arm pit of the wearer. Exemplary embodiments described herein may orient support portions and/or bias of stretch to rotate or position underlying body anatomy. For example, exemplary embodiments of the sleeve described herein may be used to rotate the arm to move material away from the axilla of the wearer or redirect forces away from the armpit of the wearer.

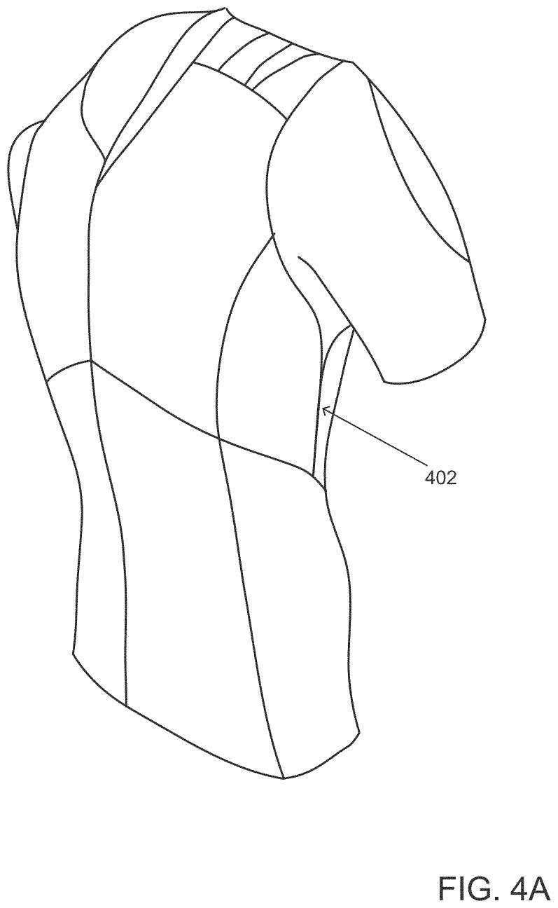

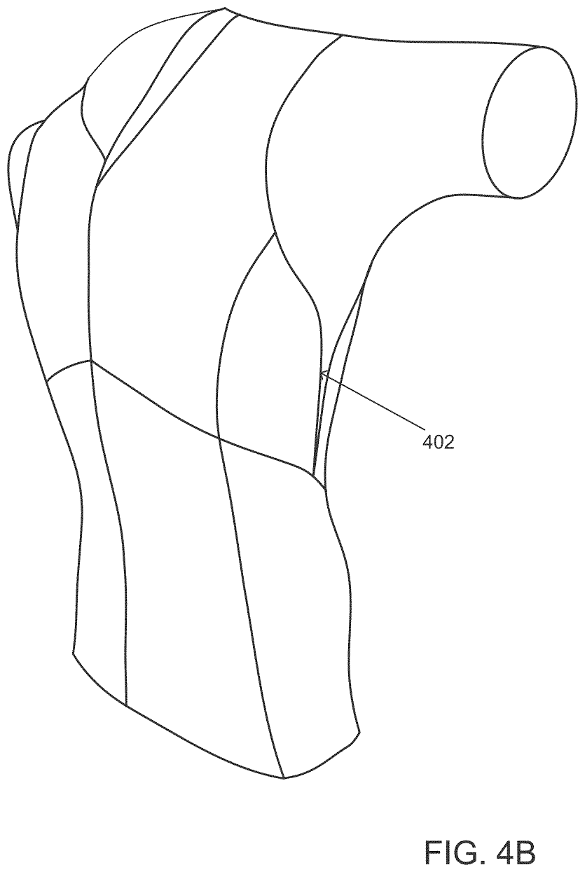

FIGS. 4A-4B illustrate an exemplary embodiment in which the arm aperture where the body portion and sleeve portion meet includes a seam 402 for diverting pulling forces of the garment away from the arm pit of the wearer. The seam attaches the sleeve to the body portion of the garment and is directed downward toward the bottom of the garment, and away from the center of the arm aperture. The seam defines an apex or point directed downward. As illustrated, the seam may extend downward to a dividing portion 304, but may also terminate along the lateral panel, or extend to the bottom of the garment. The seam 402 defines a sleeve portion configured to position under the armpit of the wearer that defines a projection down the lateral side of the garment below the arm aperture. The projection tapers from a width greatest at the top and narrows as the projection extends down the lateral side of the garment.

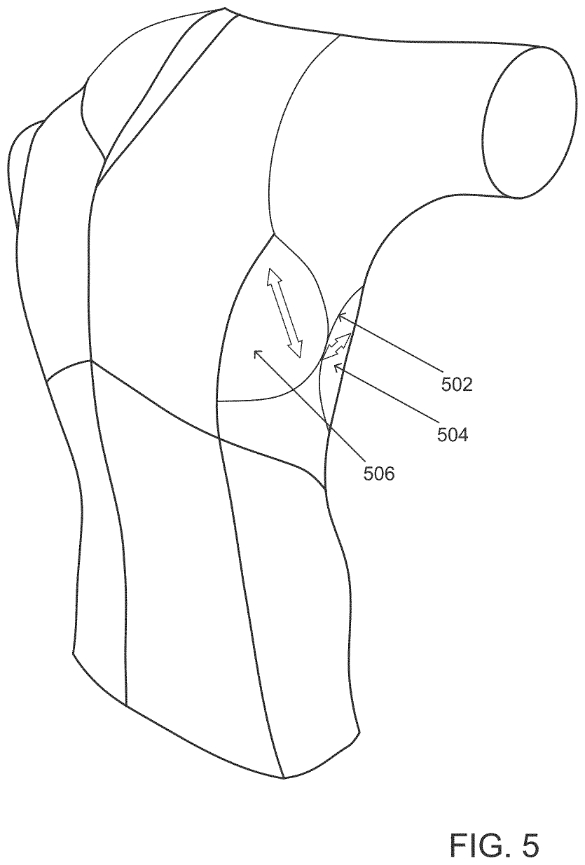

In an exemplary embodiment, seam 402 may be created or defined by different configurations of panels and/or garment portions. FIG. 5 illustrates an exemplary embodiment in which the seam 502 is created between two lateral side panels 504 and 506. For example, seam 502 similarly defines a projection of the sleeve portion that extends downward along the lateral side of the garment having a greatest width at the top and tapering to a narrower width at the bottom. The seam 502 is defined or created between two lateral side panels 504, 506. As seen the two lateral side panels define arcuate panels extending from the front and back of the garment, respectively and meet along the lateral side of the garment at the seam 502. The panels 504, 506 may include a bias of elasticity as illustrated. For example, the bias of elasticity may be oblique to the longitudinal axis of the garment. The panels may be configured such that the respective bias of elasticity are oblique or perpendicular to each other. At least one axis of bias of elasticity of each panel 504, 506 may elongate downward and toward the lateral side of the garment, toward the other panel. The panels 504, 506 may extend from the sleeve and terminate along the lateral side of the garment as illustrated. The panels 504, 506 may also terminate lower, such as at the dividing portion 304, or the bottom of the garment.

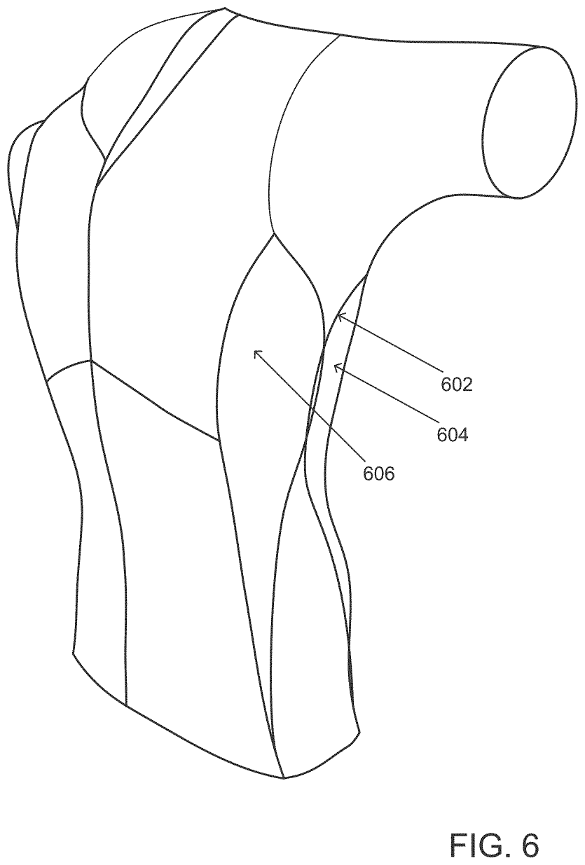

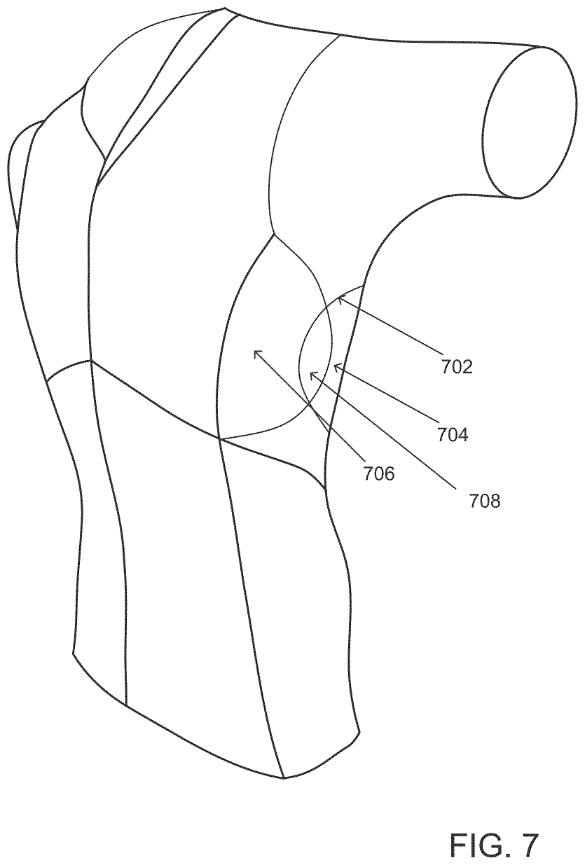

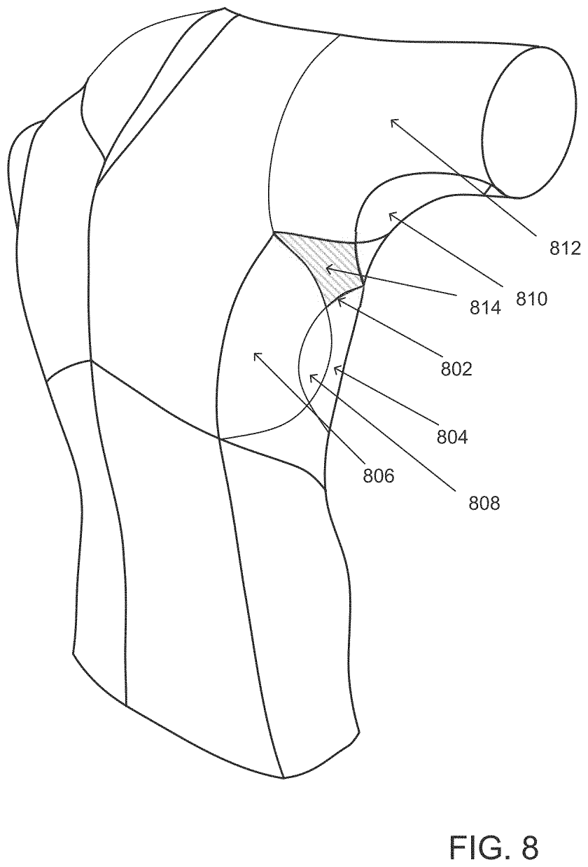

FIGS. 6-8 illustrate exemplary configurations of panels according to embodiments described herein. FIG. 6 illustrates exemplary panels in which the lateral side panels 604, 606 create a seam 602 that defines a projection from the sleeve downward along the lateral side of the garment. The projection may taper as it extends toward the bottom of the garment. The panels 604, 606 may extend to the bottom or near the bottom of the garment, as illustrated. FIG. 7 illustrates an exemplary panel configuration in which panels from opposing sides of the garment overlap to define a reduced area of elasticity at the overlap portion under the arm of the wearer in the in use configuration. As illustrated, the seam 702 defines a projection extending downward, along the latera side of the garment, toward the bottom edge of the garment. The projection is defined, in part, on opposing sides by panels 704, 706. The panels 704, 706 overlap at section 708 to define a reduced elasticity section as compared to panels 704, 706. The overlap is a dual or multi-layered area to assist in diverting the forces imposed on the garment during use and/or movement out of the arm pit of the wearer. FIG. 8 illustrates an exemplary embodiment having a seam 802 defined by the terminal edge of the panels 804, 806, but not coupled to another portion defining the sleeve. Instead, a separation or gap 814 is created between sleeve 812, 810 and lateral side panels 804, 806. The sleeve may also be created by overlapping the material of the sleeve similar to the overlap in lateral side panels. Any combination of side panels and configurations may be used and are within the scope of the instant invention. For example, the gap 814 may be included in any of the embodiments described herein. Different combination of features, such as the length, position, orientation, overlap, separation, etc. may be used to create a garment according to embodiments described herein.

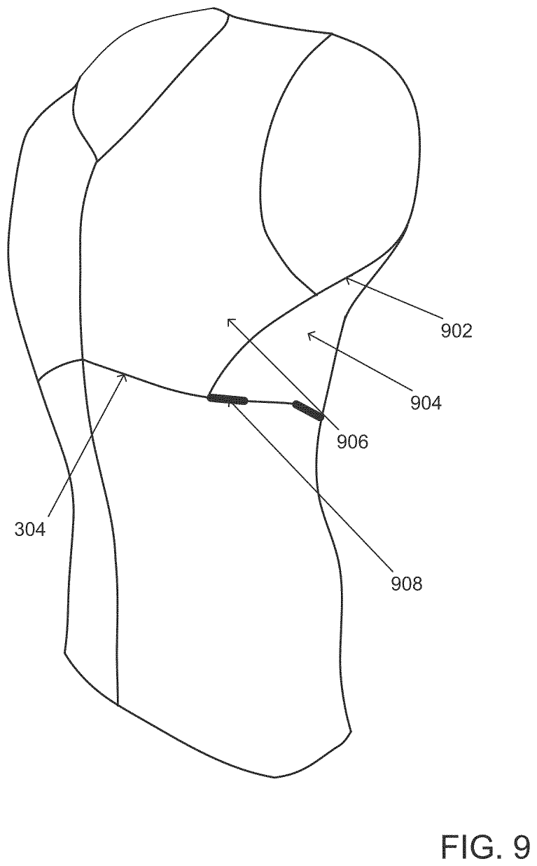

FIG. 9 illustrates an exemplary sleeve configuration according to embodiments described herein. As illustrated, an arm aperture is created by seam 902, in which seam 902 may be an exterior edge of one or more panels or garment sections according to embodiments described herein. Arm aperture and/or seam 902 may be connected to or not connected to a sleeve. The seam may therefore be a terminal end of the garment. As illustrated, the arm aperture is configured to extend downward and taper toward a lower edge of the garment. The arm aperture is created by an overlapping of panels 904, 906. The panels 904, 906 may be an extension of the same panel extending around the garment or may be separate panels coupled together. The overlap may extend to dividing portion 304 or be positioned elsewhere on the garment. The panels may be free to move relative to each other at the overlap portion. Therefore, the panels 904, 906 may not be coupled, connected, or attached along a portion of the overlap seam, such as along the edge along the top of panels adjacent or proximate to the arm aperture. The panels 904, 906 may be attached to the garment along a bottom edge or one or more points along the bottom edge of the panels, such as along dividing portion 304. Exemplary attachment of panels 904, 906 to garment at the overlapping area are indicated by darker lines 908. The back panel 904 may therefore be pulled or moved along the edge of the arm aperture independent of the position or minimizing the effect on the front panel 908.

In an exemplary embodiment, one or more seams of the garment may be non-woven seams. The seam may be created by overlapping panels or portions of the garment and to each other. The adhesion may be through an infusion of an elastic material into the material of the neuroband. Exemplary embodiments include an elastic seam. The elastic seam may be creating through impregnating an elastic into the material, by adhering elastic materials, by using elastic materials, and combinations thereof.

Exemplary embodiments of the garments described herein including materials having a bias of elasticity. Exemplary materials include weft/warp weave to create patterns and/or influence the bias of elasticity of the materials. For example, a Jacquard weaves may be used to introduce extra thread(s) into the design to create a system of elasticity according to embodiments described herein. Exemplary embodiments may include knit or woven fabrics. Exemplary embodiments include polyester, nylon, or combinations thereof.

In an exemplary embodiment, a garment may include a back portion made up of a plurality of panels according to embodiments described herein. Exemplary panels may include a bidirectional bias of elasticity. The bidirectional bias of elasticity may be generally outwardly away from the spine of the wearer. In an exemplary embodiment, the bias of elasticity is oblique or angled relative to the vertical orientation (along the spine) of the garment. The bidirectional bias of stretch may be perpendicular or may be oblique. On the front of the garment, the bias of elasticity may be greater on a lower portion of the garment than on an upper portion of the garment.

Exemplary embodiments include a system of using exemplary embodiments described herein. Exemplary embodiments may use, place, or adjust the location and elasticity of panels to create a form fitting posture garment corresponding to a specific user. Exemplary embodiments may include positioning a posture garment according to embodiments described herein on a portion of a body part of a user, such as the torso, arm, leg, etc. For positioning of a shirt garment over the torso of a user, the garment may be positioned over the hips of the user.

Exemplary embodiments of panels having desired elasticity characteristics are described herein. Exemplary embodiments of garment may include any combination of features as described herein. For example, an opening of one embodiment may be combined with one or more panel arrangements of another embodiment. The panels, seams, portions, features, etc. may be combined in any combination as would be understood by a person of skill in the art and the selected configurations are provided for illustration purposes only.

What is claimed is:

1. A garment, comprising:
an upper portion having first elasticity characteristics; and
a lower portion having second elasticity characteristics.

2. The garment of claim 1, wherein the lower portion has a first bias of elasticity greater than a bias of elasticity of the upper portion.

3. The garment of claim 1, wherein the upper portion has a first bias of elasticity with a major axis of a maximum of a bias of elasticity in a first direction, and the lower portion has a second bias with a major axis of a maximum of a bias of elasticity in a second direction, the first direction different than the second direction.

4. The garment of claim 3, wherein the first direction is vertical along a direction from a top of the garment to the bottom of the garment and the second direction is horizontal across the garment.

5. The garment of claim 1, further comprising a zipper.

6. The garment of claim 1, wherein the zipper is bidirection.

7. The garment of claim 1, wherein the upper portion and lower portion are separated by a dividing portion.

8. The garment of claim 7, wherein the dividing portion is curved, concave downward toward a bottom of the garment.

9. A garment comprising an arm aperture, the arm aperture creating a projection extending downward, along a lateral side of the garment.

10. The garment of claim 9, wherein a sleeve is attached to the arm aperture.

11. The garment of claim 10, wherein a gap is created in the projection of the aperture and the sleeve.

12. The garment of claim 9, wherein the aperture is defined by two panels contacting on the lateral side of the garment.

13. The garment of claim 12, wherein the panels overlap.

14. The garment of claim 9, wherein the arm aperture is created in part by an overlap of panels.
